(19) United States
(12) Patent Application Publication
(10) Pub. No.: US 2020/0123592 A1
(43) Pub. Date: Apr. 23, 2020

(54) SINGLE MOLECULE DETECTION AND QUANTIFICATION OF NUCLEIC ACIDS WITH SINGLE BASE SPECIFICITY

(71) Applicants: Quanterix Corporation, Billerica, MA (US); DestiNA Genomics Ltd., Edinburgh (GB)

(72) Inventors: Juan J. Díaz-Mochón, Armilla (Granada) (ES); David C. Duffy, Arlington, MA (US); Salvatore Pernagallo, Armilla (Granada) (ES); Hugh llyine, Edinburgh (GB); David M. Rissin, Somerville, MA (US); Barbara López-Longarela, (Armilla) Granada (ES)

(73) Assignees: Quanterix Corporation, Billerica, MA (US); DestiNA Genomics Ltd., Edinburgh (GB)

(21) Appl. No.: 16/616,134

(22) PCT Filed: May 29, 2018
(86) PCT No.: PCT/US18/34870

§ 371 Date: Nov. 22, 2019

Related U.S. Application Data
(60) Provisional application No. 62/512,450, filed on May 30, 2017.

(51) Int. Cl. C12Q 1/6806; C12Q 1/6837; C12Q 1/6886; G01N 21/64

(52) U.S. Cl. CPC C12Q 1/6806; C12Q 1/6837; G01N 2021/6439; G01N 21/6428; C12Q 1/6886

(57) ABSTRACT

Methods for detecting nucleic acids, particularly small nucleic acids such as microRNAs, involving the use of a peptide nucleic acid (PNA) probe that lacks a base and a labelled modified base corresponding to the omitted base in the PNA probe. A complex containing the target nucleic acid, the PNA probe, and the modified base can be determined using a single molecule array assay.

### CROSS-REFERENCE TO RELATED APPLICATIONS

This application claims the benefit under 35 U.S.C. § 119(e) of U.S. provisional application No. 62/512,450, filed May 30, 2017, the contents of which are incorporated by reference herein in their entirety.

### BACKGROUND OF THE INVENTION

The sensitive detection of specific sequences of nucleic acids (NA) has become an indispensable tool in biological research, drug development and the diagnosis and treatment of diseases. The field of molecular diagnostics—where detection of specific sequences enables diagnosis of cancer, infectious diseases, and hereditary diseases, as well as companion diagnostics for drugs—has emerged from these technologies. Further uses can be imagined in the fields of animal health and plant breeding. Khodakov et al. Adv. Drug Deliv. Rev., 2016, 105, 3-19; and Smith et al., J. Am. Chem. Soc., 2017, 139, 1020-1028. In recent years, the use and measurement of short sequences (<100 bases) of RNA, in particular, has proven useful for greater understanding and control of biological systems. Poliseno et al., Nature, 2010, 465, 1033-1038; and Tay et al., Nature, 2014, 505, 344-352. For example, circulating microRNAs (miRNAs) (˜22 bases) regulate gene expression have been described as valuable biomarkers for diagnosing cancer and liver diseases. Moreover, interfering RNA has been used as a therapeutic via gene silencing; and, RNA probes are the basis of gene editing techniques, such as CRISPR/Cas9.

The polymerase chain reaction (PCR) and next generation sequencing (NGS) technologies dominate the detection of NA. Khodakov et al., 2016. These methods have limitations in detecting NA, in particular short strands of RNA. Detection of short sequences is particularly challenging for assays that require multiple binding molecules, e.g., primers of PCR or sandwich binding assays. This requirement often necessitates the elongation of the target molecule using either a ligation step with an extension sequence so that the probes can bind, or use of poly(A) polymerase to poly-adenylated target miRNAs, so they can be converted into cDNA. PCR also suffers from bias, sample contamination from production of high concentration of amplicon, and requires laborious sample preparation. Techniques that address the challenges of existing assays, especially for short sequences of RNA, but maintain the high analytical sensitivity and high sequence specificity of PCR are needed, if microRNAs are to fulfill their promise as clinically valuable biomarkers in drug development and detection of cancers and disease.

It is hence of great interest to develop sensitive and specific assay methods for detecting and quantifying short nucleic acid molecules, such as microRNAs.

### SUMMARY OF THE INVENTION

The present disclosure is based on the development of an efficient and specific method for detecting nucleic acids having specific sequences such as microRNAs based on the hybridization and labeling of single nucleic acid molecules using a single immobilized probe, without the use of amplification by polymerases. This probe allows the capture of specific target sequences and discrimination of these sequences with single base specificity. This method has been successfully applied to directly detect microRNA miR122 in human serum without the use of polymerase amplification. MiR122 is a regulatory supported biomarker of liver toxicity, in serum.

Accordingly, one aspect of the present disclosure provides a method for detecting a nucleic acid of interest in a sample, comprising: (i) providing a sample suspected of containing a nucleic acid of interest; (ii) providing a plurality of beads, on which a peptide nucleic acid (PNA) probe is immobilized, wherein the PNA probe comprises a nucleotide sequence that is complementary to the nucleic acid of interest or a portion thereof, and wherein the PNA probe lacks a base at a position corresponding to a target nucleotide in the nucleic acid of interest; (iii)

incubating the sample with the plurality of beads under conditions allowing for formation of a nucleic acid/PNA complex; (iv) contacting the plurality of beads with a modified base that is complementary to the base of the target nucleotide in the nucleic acid of interest, wherein the modified base is conjugated to a first binding moiety and modified with a chemical group capable of reversible covalent reactions that reacts with the PNA probe; (v) contacting the plurality of beads with a second binding moiety that binds the first binding moiety and comprises a detectable label; and (vi) determining the fraction of beads associated with at least one detectable label, which is indicative of the concentration of the nucleic acid of interest in the sample. In some examples, the first binding moiety comprises biotin and the second binding moiety comprises streptavidin. In some examples, the PNA probe has a backbone with glutamic side chains. In some examples, steps (iii) and step (iv) may be performed simultaneously.

In another aspect, the present disclosure provides a method for detecting a nucleic acid of interest in a sample, comprising: (i) providing a sample suspected of containing a nucleic acid of interest; (ii) providing a plurality of beads, on which a peptide nucleic acid (PNA) probe is immobilized, wherein the PNA probe comprises a nucleotide sequence that is complementary to the nucleic acid of interest or a portion thereof, and wherein the PNA probe lacks a base at a position corresponding to a target nucleotide in the nucleic acid of interest; (iii) incubating the sample with the plurality of beads under conditions allowing for formation of a nucleic acid/PNA complex; (iv) contacting the plurality of beads with a modified base that is complementary to the base of the target nucleotide in the nucleic acid of interest, wherein the modified base is conjugated to a first binding moiety and modified with a chemical group capable of reversible covalent reactions that reacts with the PNA probe; (v) contacting the plurality of beads with a second binding moiety that binds the first binding moiety; (v) contacting the plurality of beads with a third binding moiety that binds the second binding moiety and comprises a detectable label; and (vii) determining the fraction of beads associated with at least one detectable label, which is indicative of the level of the nucleic acid of interest in the sample. In some examples, the first binding moiety can be a fluorescein label. In some examples, the second binding moiety comprises an antibody specific to the first binding moiety, for example, an antibody binding to a fluorescein label. In some instances, the antibody can be biotinylated and the third binding moiety comprises streptavidin.

In some examples, the first binding moiety can be a maleimide group. In some examples, the second binding moiety comprises a biotinylated heterocarbon chain with a thiol group that reacts specifically with the first binding moiety. In some instances, the heterocarbon chain has 1-10 biotin and the third binding moiety comprises streptavidin. In some examples, the second binding moiety comprises nucleic acid with a thiol group that reacts specifically with the first binding moiety. In some instances, the second binding moiety binds to multiple complementary nucleic acids comprising a biotin label that then bind to streptavidin.

In some embodiments, the detectable label used in any of the assay methods described herein may release a signal directly, for example, a dye or a fluorescent agent. In some embodiments, the detectable label may release a signal indirectly, for example, a β-galactosidase, which may convert a fluorogenic substrate to a product that generates a fluorescent signal.

In any of the assay methods described herein, the nucleic acid of interest is a microRNA, e.g., a microRNA associated with a disease. The sample to be analyzed by the assay method may be a biological sample obtained from a subject, for example, a subject suspected of having the disease. In some instances, the chemical group in the modified base can be an aldehyde group, a ketone group, a thiol group, or a diol group capable of reversible covalent reactions. In some examples, the PNA probe has 10-50 monomer units (e.g., 10-20, 10-30, or 10-40 monomer units).

In any of the assay methods, the fraction of beads associated with at least one detectable label may be detected using a single molecule array, for example, a Simoa assay.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

FIG. 1 is a schematic illustration of an exemplary approach for detecting single molecules of nucleic acid (NA) with single base specificity. Panel I illustrates the reaction when a capture probe has a perfect match with a target NA. Panel II illustrates the reaction when a capture probe has one mismatched base with a target NA. Panel III illustrates the reaction when a capture probe does not match a target NA.

FIG. 2 is a diagram showing Plot of AEB determined using the assay illustrated in FIG. 1 against concentration of calibrators for miR-122 (circles) and miR-122 with a single base mismatch at the 9th position (squares) spiked into buffer. The sequences of these two molecules are shown in Table 4. Error bars (+1 s.d.) are smaller than the size of the data point.

FIG. 3 includes diagrams showing measurement of miR122 in clinical samples. A: Scatter plot of AEB for miR-122 measured in the serum of 4 healthy volunteers and 4 individuals after overdosing on acetominophen. The dotted lines represent the mean assay (buffer) background±1 s.d. B: Correlation of concentration of miR-122 in the serum of patients determined using Simoa and PCR (r2=0.93; slope=0.64).

FIG. 4 includes diagrams showing structures of compounds used in an exemplary assay. A: structure of an exemplary Amino-PEG linker (“xx” in Table 1) located at the N-terminal end of the abasic PNA probe (1). B: Structure of aldehyde-modified cytosine base presenting biotin.

FIG. 5 is a plot showing ES+-TOF MS of base, m/z 1036.5806 [M+H]+; 1054.5632 [M+H2O+H]⁺; 1076.5531 [M+H2O+Na]⁺; 518.7623 [M+2H]⁺²; 538.7747 [M+H2O+Na+H]⁺²; High Resolution Mass Spectroscopy (HRMS) calculated for C46H82N7O17S ([M+H]⁺⁾1036.5482, found 1036.5806. Hydrated form of aldehyde is detected.

FIG. 6 is a plot of Ct values determined using PCR as a function of the concentration of miR-122 (see Table 4). To estimate the limit of detection, the Ct values of the three lowest concentrations were averaged and the standard deviation determined. The Ct value at 3 s.d. below the mean Ct was 34, so the LOD was about 2.6 fM. Error bars are shown as 1 s.d.

FIG. 7 is a diagram showing specificity of Simoa assay for miR-122. The chart shows AEB values of the miR-122 specific beads for: 15 nM miR-122 calibrator (1st bar); 15 nM of miR-122 calibrator with a single base mismatch (2nd bar); 15 nM of miR-39 (3rd bar); no nucleic acid (4^{th} bar); no nucleic acid and no nucleobase label (5th bar); no nucleic acid and no reductant (6th bar); and, no nucleic acid, no nucleobase, and no reductant (7th bar). The last 4 bars in the chart are reagent drop-out experiments that indicate the sources of background in the assay. These data indicate the non-specific binding of the nucleobase label to the beads dominates the background of the Simoa assay. Error bars are s.d. values from 2 replicates. Each bar is labelled with its AEB value.

FIG. 8 is a diagram showing Ct values for patients and healthy controls for the same samples measured using Simoa as described in FIG. 3, panel A.

FIG. 9 is a diagram showing the correlation of concentration of synthetic calibrator for miR-122 spiked into serum determined using Simoa and PCR (r²=0.998).

FIG. 10 is a diagram showing a Bland-Altman plot for concentrations of miR-122 in serum samples of patients as described herein determined using Simoa and PCR. The dashed line is at the mean of the difference in concentration between the methods; the dotted lines are at +1.96 s.d. of the mean. Bland et al., Lancet 1986, 1, 307-10.

### DETAILED DESCRIPTION OF THE INVENTION

Described herein is a single probe method for detecting single molecules of a nucleic acid of interest (e.g., a small nucleic acid such as a microRNA) from a suitable biological sample (e.g., human serum), with sequence specificity down to a single base. The method described herein may not require amplification of the nucleic acid of interest. The assay methods described herein involve a combination of a single-base label approach and a detection assay capable of counting single molecules/single labels. The single-base label approach has been used to demonstrate gentotyping assays using mass spectrometry, DNA microarrays, and conventional bead-based assays. Bowler et al., Angew. Chem. Int. Ed. 2010, 49:1809-1812; Pernagallo et al., Sensors, 2012, 12:8100-8111; and Venkateswaran et al., Talanta, 2016, 161:489-496. The sensitivity of these analog assays were, however, not sufficient to detect miRNA in clinical samples. Venkateswaran et al., Talanta, 2016, 161:489-496. It was demonstrated that single molecule arrays (Simoa) are very sensitive to an enzyme label (limit of detection, LOD=220 zM), and could be used to develop sensitive assays for proteins and DNA. Rissin et al., Nat. Biotechnol. 2010, 28:595-599; and Song et al., Anal. Chem. 2013, 85:1932-1939. The Simoa DNA assay was, however, limited to relatively long target sequences (>100 base pairs) because of the requirement for a capture and multiple detection probes, each being 15-20 bases long.

Combining these two approaches enables an assay with unexpectedly high sensitivity (e.g., single molecule) and high specificity (e.g., single base) for detecting short RNA target molecules, such as microRNAs.

### I. Methods for Detecting Nucleic Acids Involving Single Base Labeling and Single Molecule/Single Label Detection

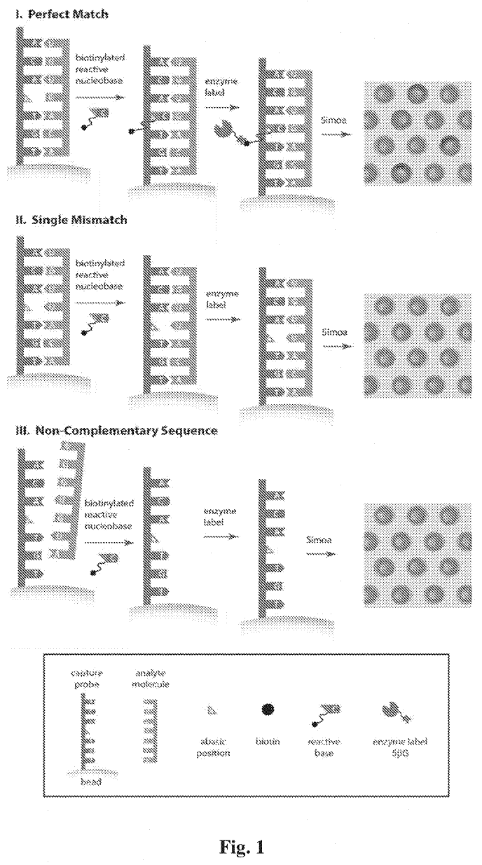

FIG. 1 illustrates an example of the methods described herein for detecting target nucleic acids, particularly small nucleic acids such as microRNAs, with high sensitivity and specificity (e.g., single base specificity and single molecule sensitivity). The exemplary approach combines the specific labeling of an immobilized capture probe with a biotinylated single nucleobase, with detection in arrays of femtoliter wells of single enzymes that bind to the biotin.

In some embodiments, peptide nucleic acid (PNA) probes are used in the assay methods described herein. PNAs are synthetic polymers mimicking the structures of DNAs or RNAs. As used herein, the term “PNA probe” refers to any synthetic polymer that mimics the structures of DNAs or RNAs and has a peptide backbone, which may include one or more modification moieties. In some instances, the backbone of a PNA molecule is composed of repeating N-(2-aminoethyl)-glycine units linked by peptide bonds, rather than the deoxyribose or ribose sugar backbones in DNAs or RNAs. A PNA molecule contains various purine and/or pyrimidine bases, just like DNAs or RNAs, which are linked to the backbone by a methylene bridge (—CH₂—) and a carbonyl group (—(C═O)—). Such molecules are depicted like peptides, with the N-terminus at the first (left) position and the C-terminus at the last (right) position. The order of the purine and pyrimidine bases from the N-terminus to the C-terminus represents the nucleotide sequence of a PNA molecule, which can form a duplex with a nucleic acid carrying a sequence that is complementary (completely or partially) to the PNA molecule.

Typically, the PNA monomer from which the PNA oligomer is derived, or one or more repeat units of the PNA oligomer, is/are derived from a N-(2-aminoethyl)-glycine unit. As stated, the PNA monomer or one or more of the repeat units of the PNA oligomer may comprise at the/their gamma position(s) a charged moiety, or a moiety capable of carrying a charge at a predetermined pH.

The PNA probes described herein include modified PNA probes, for example, those having a modified backbone. In some examples, the modified PNA probes comprise a N-(2-aminoethyl)-glycine unit. Such modified PNA probes may have the general formula of:

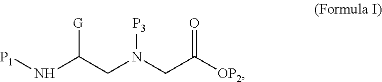

G is a charged moiety (e.g., a glutamic group), or a moiety capable of carrying a charge at a predetermined pH;

P1 is a protective group P, or is hydrogen;

P2 is a protective group P, or is hydrogen, or is a group selected from the list consisting of alkyl, cycloalkyl, aryl, aralkyl, or halogen,

P3 is hydrogen, or is a protective group P, or is a group represented by formula (II) below:

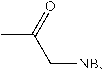

in which NB is a nucleobase. In some examples, the modified PNA probes have a backbone modified by glutamic side chains.

The PNA probes as described herein may have the general formula:

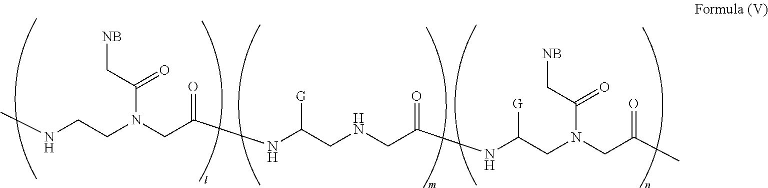

In Formula (V), G is a charged moiety, or a moiety capable of carrying a charge at a predetermined pH; NB is a nucleobase; and l≥0; m≥0; n≥0, with the proviso that l+m+n≥2 and n+m≥1. Preferably, l≥1; m≥1 (e.g., m=1); and/or n ≥1. In some embodiments, G is a glutamic group.

It will be understood that the repeat units of the PNA oligomer of formula (V) may not necessarily be provided sequentially or as a block polymer of blocks 1, m, and n, but that the repeat units may be provided in any order.

In some examples, the total number of PNA units (l+m+n) in the oligomer may be in the range of 5-50, e.g., 7-40, e.g. 10-30. In some examples, the total number of PNA units can be in the range of 12-24.

Other descriptions of PNA probes, including modified PNA probes, can be found in ES201630948 (filed 12 Jul. 2016) and GB1616556.5 (filed 29 Sep. 2016), the relevant disclosures of which are hereby incorporated by reference for the purposes or subject matter referenced herein.

The PNA probes as described herein may contain up to 50 monomer units (e.g., up to 40, 30, 25, 20, 15, or 10 monomer units). Such probes are complementary to a target nucleic acid or a portion thereof. In some instances, the PNA probes are complementary to a small nucleic acid (e.g., having less than 50 nucleotides, less than 40 nucleotides, less than 30 nucleotides, or less than 20 nucleotides). In some instances, the PNA probe used in an assay method described herein contains less than 20 monomer units. In one specific example, the PNA probe is complementary to a microRNA or a portion thereof, for example, a microRNA that is associated with a disease or disorder. Such target microRNAs are well known in the art.

“Complementary,” as used herein, refers to the nucleobase complementarity commonly known in the art. For example, adenine is complementary to thymine (in DNA) or uracil in RNA; and guanine is complementary to cytosine. “Sequence complementarity”, or “nucleic acid sequences being complementary to one another”, as used herein, means when the two nucleic acid molecules are aligned antiparallel to each other (the 5′-end of a nucleic acid sequence faces the C-terminus of a PNA sequence), the nucleotide bases at each position, or at most positions in the sequences are complementary, and that the two nucleic acid molecules can hybridize and form a duplex under suitable conditions, e.g., hybridization temperature. As known in the art, a sequence complementarity needs not be 100% for the two nucleic acid molecules to hybridize and form a duplex. The sequence complementarity between the capture probe (or the detecting probe described herein) and the target nucleic acid may be at least 80% complementary to the corresponding region in the target nucleic acid. In some embodiments, the capture probe contains a fragment that is at least 80% (e.g., 85%, 90%, 95%, 98%, or 100%) complementary to the first segment of the target nucleic acid. In some instances, the capture probe contains a fragment that is completely complementary (100% complementary) to the first segment of the target nucleic acid. Such a capture probe may be used in differentiating the target nucleic acid from substantially similar nucleic acids, for example, nucleic acids having 1, 2, or 3 base differences relative to the target nucleic acid.

As illustrated in FIG. 1, the PNA probe described herein is an abasic PNA probe, i.e., lacking one base at a position corresponding to a target nucleotide in the nucleic acid of interest (the nucleic acid to be detected using the PNA probe), for example, a microRNA. The position where the base is lacking can be any position within the PNA probe. In some instances, the base-missing position may be located in the middle of the PNA probe, for example, having at least three bases at the N-terminal, at the C-terminal, or both. The target nucleotide may be at any position inside the nucleic acid of interest. In some instances, the target nucleotide is distinctive to the nucleic acid of interest, e.g., presented in the nucleic acid of interest but not in homologous nucleic acids. For example, if the nucleic acid of interest is a microRNA that belongs to a family containing structurally similar members, the target nucleotide can be located at a position that can be used to distinguish the microRNA of interest from other members of the same family.

The PNA probe as described herein can be immobilized on a support member via a conventional method. As used herein, “immobilized” means attached, bound, or affixed, covalently or non-covalently, so as to prevent dissociation or loss of the capture probe, but does not require absolute immobility with respect to either the capture probe or the support member. A support member can be a solid or semi-solid member with a surface that can be used to specifically attach, bind or otherwise capture a nucleotide probe (e.g., the capture PNA probe of the present disclosure), such that the PNA probe becomes immobilized with respect to the support member. In some instances, the PNA probe is immobilized onto a support member (e.g., a bead) directly through a covalent bond (e.g., amide, disulfide, hydrazine or thioether). In other instances, the PNA probe may be immobilized onto a support member (e.g., a bead) via linker, such as an oligonucleotide linker (e.g., a polyA or poly T linker) or a peptide linker (e.g., a poly glycine or poly alanine linker).

The support member of the present disclosure may be fabricated from one or more suitable materials, for example, plastics or synthetic polymers (e.g., polyethylene, polypropylene, polystyrene, polyamide, polyurethane, phenolic polymers, or nitrocellulose), naturally derived polymers (e.g., latex rubber, polysaccharides, polypeptides), composite materials, ceramics, silica or silica-based materials, carbon, metals or metal compounds (e.g., comprising gold, silver, steel, aluminum, or copper), inorganic glasses, silica, and a variety of other suitable materials. Non-limiting examples of potentially suitable configurations include beads (e.g., magnetic beads), tubes (e.g., nanotubes), plates, disks, dipsticks, chips, microchips, coverslips, or the like. In one particular example, the support member is a bead.

The surface of the support member of the present disclosure may comprise any molecule, other chemical/biological entity, or solid support modification disposed upon the solid support that can be used to specifically attach, bind or otherwise capture a PNA molecule. Surface compositions that may be used to immobilize a PNA molecule can be readily found in the art. For example, the surface may comprise a complementary nucleic acid or a nucleic acid binding protein, which can be attached to the surface via convention methods. Thus, the linkage between the PNA to be immobilized (e.g., the capture probe of the present disclosure) and the surface may comprise one or more chemical or physical (e.g., non-specific attachment via van der Waals forces, hydrogen bonding, electrostatic interactions, hydrophobic/hydrophilic interactions; etc.) bonds and/or chemical linkers providing such bond(s). Alternatively, the surface of the support member may comprise reactive functional groups that are capable of forming covalent bonds with the nucleic acid molecules to immobilize. In some embodiments, the functional groups are chemical functionalities. That is, the binding surface may be derivatized such that a chemical functionality is presented at the binding surface, which can react with a chemical functionality on the nucleic acid to be captured, resulting in attachment. Examples of functional groups for attachment that may be useful include, but are not limited to, amino groups, carboxyl groups, epoxide groups, maleimide groups, oxo groups, azides and thiol groups. Functional groups can be attached, either directly or through the use of a linker, the combination of which is sometimes referred to herein as a “crosslinker.” Crosslinkers for attaching nucleic acid molecules to a support member are known in the art; for example, homo-or hetero-bifunctional crosslinkers as are well known (e.g., see 1994 Pierce Chemical Company catalog, technical section on crosslinkers, pages 155-200, or “Bioconjugate Techniques” by Greg T. Hermanson, Academic Press, 1996). Non-limiting example of crosslinkers include alkyl groups (including substituted alkyl groups and alkyl groups containing heteroatom moieties), esters, amide, amine, thiols, azides, triazine, epoxy groups and ethylene glycol and derivatives. A linker may also be a sulfone group, forming a sulfonamide. In some embodiments, the functional group is a light-activated functional group. That is, the functional group can be activated by light to attach the capture component to the capture object surface. One example is PhotoLink™ technology available from SurModics, Inc. in Eden Prairie, Minn.

It is to be understood that the examples provided herein on the support member and the surface composition are not meant to be limiting. Any support members that are known in the art to be suitable for immobilization of nucleic acid molecules may be used in accordance with the present disclosure.

A suitable PNA probe, optionally immobilized on a support member, can be incubated with a sample suspected of having a nucleic acid of interest under suitable conditions to allow hybridization of the PNA probe and the nucleic acid having a complementary sequence to the PNA probe, which lead to formation of PNA/nucleic acid duplexes. See FIG. 1. Hybridization refers to the ability of complementary single-stranded DNA/RNA and the PNA probe to form a PNA/nucleic acid duplex molecule. The hybridization step of the assay method described herein can be performed under suitable hybridization conditions, which are within the knowledge of those skilled in the art. Hybridization conditions resulting in particular degrees of stringency will vary depending upon the nature of the hybridization method and the composition and length of the hybridizing nucleic acid sequences. Generally, the temperature of hybridization and the ionic strength of the hybridization buffer will determine the stringency of hybridization. Calculations regarding hybridization conditions for attaining particular degrees of stringency are well known in the art, for example, described in Sambrook et al., (1989) Molecular Cloning, second edition, Cold Spring Harbor Laboratory, Plainview, N.Y. (chapters 9 and 11). The hybridization temperatures of the first and second hybridization steps of the assay methods described herein can be determined based on various factors, for example, the length of the complementary regions between the capture/detecting probe and the target nucleic acid, the composition of the complementary regions (e.g., G/C content), and the stringency needed, which are within the knowledge of those skilled in the art.

When needed, the hybridization reaction mixture described above can be washed one or more times to remove unbound nucleic acids and other components so as to enhance sensitivity and specificity of the assay method. Any nucleic acid having a complementary nucleotide sequence of the PNA probe will form a duplex attached to the support member (e.g., the bead). As illustrated in FIG. 1, nucleic acids having completely complementary sequences or partially complementary sequences would hybrid with the PNA probe to form a duplex, while nucleic acids having non-complementary sequences would not form duplex with the PNA probe.

During or after hybridization, the mixture containing the PNA/nucleic acid duplex (if any) can be incubated with a modified base, which is complementary to the target nucleotide in the nucleic acid of interest. The modified base contains a chemical group that is capable of reacting with the PNA probe for form a covalent bond. Any modified bases capable of reacting with a PNA molecule can be used in the instant assay methods, for example, those described in US2011/0028337, the relevant disclosures of which are incorporated by reference herein for the purposes or subject matter referenced herein. In some instances, the functional group in the modified base can be a group capable of reversible covalent reactions such as an aldehyde group, a ketone group, a thiol group, or a diol group. The modified base is also attached to a first binding moiety, which may be a label capable of releasing a signal directly or indirectly (a detectable label), or a member of a ligand/receptor pair. In one example, the first binding moiety can be biotin. Any methods known in the art involving dynamic labeling chemistry to specifically label the PNA probe bound to the complementary nucleic acid of interest with any of the modified bases described herein can be used in the present disclosure, for example, the method described in Bowler et al., Angew. Chem. Int. Ed. 2010, 49:1809-1812, the relevant disclosures of which are incorporated by reference herein for the purposes or subject matter referenced herein.

Afterwards, the reaction mixture may be washed one or more times to remove unbound modified bases. The PNA/nucleic acid duplex labeled with the modified base may optionally be incubated with a second binding moiety which specifically binds the first binding moiety and comprises a detectable label, which may be measured directly.

In some instances, the first binding moiety incorporated into the modified base and the second binding moiety are members of a ligand/receptor pair, for example, biotin and streptavidin. In some instances, the streptavidin is conjugated to a detectable label, e.g., an enzyme, such as beta-galactosidase. In other instances, the second binding moiety may be an antibody or an antigen-binding fragment thereof that specifically binds the first binding moiety.

In some embodiments, the second binding moiety may be an antibody (e.g., an anti-fluorescein antibody) or an antigen-binding fragment thereof that specifically binds the first binding moiety (e.g., a fluorescein molecule). The second binding moiety may be conjugated to an agent (e.g., biotin) that specifically binds a third binding moiety (e.g., streptavidin), which is conjugated to a detectable label.

In some embodiments, the second binding moiety may be a heterocarbon chain containing a thiol groups thereof that specifically binds the first binding moiety (e.g., a maleimide group). The second binding moiety may be conjugated to an agent or multiple agents (e.g., biotin) that specifically binds a third binding moiety (e.g., streptavidin), which is conjugated to a detectable label.

In some embodiments, the second binding moiety comprises nucleic acid with a thiol group that reacts specifically with the first binding moiety. In some instances, the second binding moiety binds to multiple complementary nucleic acids comprising a biotin label that then bind to streptavidin. “Conjugated”, as used herein, means an agent (e.g., a detectable label) is attached to another molecule (e.g., a binding moiety as described herein) the detecting probe, covalently or non-covalently. The detectable label can be any molecule, particle, or the like, that facilitates detection, directly or indirectly, using a suitable detection technique.

In some embodiments, the detectable label may be a molecule or moiety capable of releasing a signal that can be directly interrogated and/or detected (e.g., a fluorescent label or a dye). In some embodiments, a fluorescent label is used as the detectable agent. Examples include, but are not limited to, fluorescein, isothiocyanate, rhodamine, phycoerythrin, phycocyanin, allophycocyanin, o-phthaldehyde, fluorescamine and fluorescent metals such as ¹⁵²Eu or other metals from the lanthanide series, CYE dyes, SETA dyes, and fluorescent proteins such as eGFP, eYFP, eCFP, mKate2, mCherry, mPlum, mGrape2, mRaspberry, mGrape1, mStrawberry, mTangerine, mBanana, and mHoneydrew.

In other embodiments, the detectable agent may be a molecule or moiety (e.g., an enzyme) capable of converting a substrate to a product that is capable of releasing a detectable signal. For example, the detectable label may be a luciferase, which converts luciferin to oxyluciferin to emit detectable lights. Alternatively, the detectable label may be β-D-galactosidase, which can convert its substrate resorufin-3-galactopyranoside (RGP) to a product (resorufin) that has a detectable fluorescent signal.

Other exemplary detectable labels include, but are not limited to, phosphorescent labels, chemiluminescent labels or bioluminescent labels (such as luminal, isoluminol, theromatic acridinium ester, imidazole, acridinium salts, oxalate ester, and dioxetane), radio-isotopes (such as ³H, ¹²⁵I, ³²P, ³⁵S, ¹⁴C, ⁵¹Cr, ³⁶Cl, ⁵⁷CO, ⁵⁸Co, ⁵⁹Fe, and ⁷⁵Se), metals, metal chelates or metallic cations (for example metallic cations such as ^{99m}Tc, ¹²³I, ¹¹¹In, ¹³¹I, ⁹⁷Ru, ⁶⁷Cu, ⁶⁷Ga, and ⁶⁸Ga. Other examples include chromophores and enzymes (e.g., malate dehydrogenase, staphylococcal nuclease, delta-V-steroid isomerase, yeast alcohol dehydrogenase, alpha-glycerophosphate dehydrogenase, triose phosphate isomerase, peroxidase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholine esterase).

Any of the assay methods described herein employs a step of measuring the intensity of the signal released from the detectable label (directly or indirectly), which can be achieved by a detection assay capable of counting single molecules (e.g., single detectable labels), for example a single molecule array assay (e.g., SiMoA™). In some embodiments, supporting members such as beads to which a complex of PNA probe-nucleic acid of interest-binding moiety-detectable label is attached can be segregated into a plurality of locations (e.g., a plurality of wells of a multi-well plate) to facilitate detection/quantification, such that each location comprises/contains either zero or one or more detectable label molecule. In some instances, each location contains one molecule of the nucleic acid of interest (corresponding to one detectable label molecule).

Additionally, in some embodiments, the locations may be configured in a manner such that each location can be individually addressed. In some embodiments, a measure of the concentration of the nucleic acid of interest (e.g., the concentration of a microRNA of interest) in a fluid sample may be determined by detecting the nucleic acids immobilized with respect to a binding surface having affinity for at least one type of nucleic acids. In certain embodiments the binding surface may form (e.g., a surface of a well/reaction vessel on a substrate) or be contained within (e.g., a surface of a capture object, such as a bead, contained within a well) one of a plurality of locations (e.g., a plurality of wells/reaction vessels) on a substrate (e.g., plate, dish, chip, optical fiber end, etc). At least a portion of the locations may be addressed and a measure indicative of the number/percentage/fraction of the locations containing at least one molecule of the nucleic acid of interest may be made. In some cases, based upon the number/percentage/fraction, a measure of the concentration of the nucleic acid of interest in the fluid sample (e.g., the biological sample) may be determined. The measure of the concentration of the nucleic acid of interest in the fluid sample may be determined by a digital analysis method/system optionally employing Poisson distribution adjustment and/or based at least in part on a measured intensity of a signal, as will be known to those of ordinary skill in the art. In some cases, the assay methods and/or systems may be automated.

Additional details of exemplary, non-limiting assay methods, which comprise one or more steps of spatially segregating the nucleic acid of interest to be detected by any of the assay methods described herein, will now be described. In certain embodiments, a method for detection and/or quantifying a nucleic acid of interest in a sample comprises immobilizing a plurality of the nucleic acid molecules with respect to a plurality of capture objects (e.g., beads) that each include a binding surface having affinity for at least one type of nucleic acid molecules. For example, the capture objects may comprise a plurality of beads comprising a plurality of capture components (e.g., a PNA probe as described herein). At least some of the capture objects may be spatially separated/segregated into a plurality of locations, and at least some of the locations may be addressed/interrogated (e.g., using an imaging system). A measure of the concentration of the nucleic acid molecules in the fluid sample may be determined based on the information received when addressing the locations (e.g., using the information received from the imaging system and/or processed using a computer implemented control system). In some cases, a measure of the concentration may be based at least in part on the number of locations determined to contain a capture object that is or was associated with at least one molecule of the nucleic acid of interest. In other cases and/or under differing conditions, a measure of the concentration may be based at least in part on an intensity level of at least one signal indicative of the presence of a plurality of nucleic acids of interest and/or capture objects associated with a molecule of the nucleic acid of interest at one or more of the addressed locations.

In some embodiments, the number/percentage/fraction of locations containing a capture object but not containing a molecule of the nucleic acid of interest may also be determined and/or the number/percentage/fraction of locations not containing any capture object may also be determined. In such embodiments, a measure of the concentration of the nucleic acid of interest in the fluid sample may be based at least in part on the ratio of the number of locations determined to contain a capture object associated with a molecule of the nucleic acid of interest to the total number of locations determined to contain a capture object not associated with any molecule of the nucleic acid of interest, and/or a measure of the concentration of the nucleic acid of interest in the fluid sample may be based at least in part on the ratio of the number of locations determined to contain a capture object associated with a molecule of the nucleic acid of interest to the number of locations determined to not contain any capture objects, and/or a measure of the concentration of the nucleic acid of interest in the fluid sample may be based at least in part on the ratio of the number of locations determined to contain a capture object associated with a molecule of the nucleic acid of interest to the number of locations determined to contain a capture object. In yet other embodiments, a measure of the concentration of nucleic acids of interest in a fluid sample may be based at least in part on the ratio of the number of locations determined to contain a capture object and a nucleic acid of interest to the total number of locations addressed and/or analyzed.

In certain embodiments, at least some of the plurality of capture objects (e.g., at least some associated with at least one molecule of the nucleic acid of interest) are spatially separated into a plurality of locations, for example, a plurality of reaction vessels in an array format. The plurality of reaction vessels may be formed in, on and/or of any suitable material, and in some cases, the reaction vessels can be sealed or may be formed upon the mating of a substrate with a sealing component, as discussed in more detail below. In certain embodiments, especially where quantization of the capture objects associated with at least one molecule of the nucleic acid of interest is desired, the partitioning of the capture objects can be performed such that at least some (e.g., a statistically significant fraction; e.g., as described in WO 2011/109364, by Duffy et al., the relevant disclosures of which are incorporated by reference herein) of the reaction vessels comprise at least one or, in certain cases, only one capture object associated with at least one molecule of a nucleic acid of interest and at least some (e.g., a statistically significant fraction) of the reaction vessels comprise an capture object not associated with any nucleic acid of interest. The capture objects associated with at least one nucleic acid of interest may be quantified in certain embodiments, thereby allowing for the detection and/or quantification of nucleic acids of interest in the fluid sample by techniques described in more detail herein.

In some embodiments, the detection step of any of the assay methods described herein involves a single molecule array assay (for example, the SiMoA™ technology) known in the art. Exemplary single molecule array assays have been described previously, for example, U.S. Pat. Nos. 8,460,879, 8,460,878, 8,492,098, 8,222,047, 8,236,574, 8,415,171, US2010-0075862, US2010-0075439, US2010-0075355, US 2011-0212462, US 2012-0196774, US 2011-0245097, WO 2009/029073, WO2010/039179, WO2011/109364, WO2011/109372, WO2011/109379, WO/2014/113502, the relevant disclosures of each of which are incorporated by reference herein for purposes or subject matter referenced herein.

### II. Applications

Any of the assay methods described herein can be used to detect the presence and/or measure the level of a nucleic acid of interest (e.g., a microRNA such as one associated with a target disease or disorder) in a suitable sample. In some examples, the sample may be a biological sample obtained from a subject and the results obtained from the assay methods described herein may be used for diagnostic and/or prognostic purposes. In other examples, the assay methods described herein can be used in research settings for detecting presence or measuring the level of a nucleic acid of interest in a sample.

To detect the presence or measure the level (concentration) of a nucleic acid of interest in a sample, a calibration curve may be developed using samples containing known concentrations of the nucleic acid of interest. The concentration of the nucleic acid of interest in a sample may be determined by comparison of a measured parameter to a calibration standard. In some cases, a calibration curve may be prepared, wherein the total measured signal is determined for a plurality of samples comprising the nucleic acid of interest at a known concentration using a substantially similar assay format. For example, the total intensity of the array may be compared to a calibration curve to determine a measure of the concentration of the nucleic acid of interest in the sample. The calibration curve may be produced by completing the assay with a plurality of standardized samples of known concentration under similar conditions used to analyze test samples with unknown concentrations. A calibration curve may relate the detected signal of the nucleic acid of interest (and/or detecting probe) with a known concentration of the nucleic acid of interest. The assay may then be completed on a sample containing the nucleic acid of interest or fragment in an unknown concentration, and signals detected from the nucleic acid of interest (and/or detecting probe) may be compared to the calibration curve, (or a mathematical equation fitting same) to determine a measure of the concentration of the nucleic acid of interest in the sample.

The assay methods described herein may be used to detect any nucleic acid molecule and their mimics, including both DNA molecules and RNA molecules. When the target nucleic acid is a DNA molecule, a denaturing step may be performed to produce single-stranded DNA molecules, e.g., by heating the sample to 96° C. or added NaOH. In some embodiments, the assay methods are applied to detecting short nucleic acids, for example, nucleic acids having less than 150 nucleotides (nts), e.g., less than 120 nts, less than 100 nts, less than 80 nts, less than 60 nts, less than 50 nts, less than 40 nts, less than 30 nts, less than 25 nts, or less than 20 nts. In a particular example, the assay methods are applied for detecting microRNAs. Given the high sensitivity and specificity of the assay methods described herein, a nucleic acid of interest in a biological sample may not need to be pre-amplified using polymerases or other methods.

In some embodiments, the assay methods are applied to detect a nucleic acid of interest in a biological sample, which may be any sample from a biological source. Exemplary biological samples include tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, blood components (such serum and plasma), urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample. In some embodiments, the biological sample can be a body fluid, which can be fluid isolated from the body of an individual. For example, “body fluid” may include blood, plasma, serum, bile, saliva, urine, tears, perspiration, and the like. As the method does not require amplification using polymerases that are affected by biological materials found in many samples, it may be used to detect NA in samples without purification of the NA, i.e., the direct detection of NA in samples.

The biological sample may be obtained from a subject in need of the analysis. A “subject” may be a human (i.e., male or female of any age group, for example, pediatric subject (e.g., infant, child, or adolescent) or adult subject (e.g., young adult, middle-aged adult, or senior adult). Alternatively, the subject may be a non-human animal. In certain embodiments, the non-human animal is a mammal (e.g., primate, for example, cynomolgus monkey or rhesus monkey), commercially relevant mammal (e.g., cattle, pig, horse, sheep, goat, cat, or dog), or bird (e.g., commercially relevant bird, such as chicken, duck, goose, or turkey). In other examples, the non-human animal is a fish, reptile, or amphibian. The non-human animal may be a male or female at any stage of development. The non-human animal may be a transgenic animal or genetically engineered animal. In some examples, the subject may also be a plant.

In some embodiments, the sample for analysis may contain one or more nucleic acids that are highly homologous to the nucleic acid of interest, e.g., at least 80%, 90%, 95%, or 98% identical to the target nucleic acid. The “percent identity” of two nucleic acids can be determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST nucleotide searches can be performed with the NBLAST program, score=100, wordlength-12 to obtain nucleotide sequences homologous to the nucleic acid molecules of the invention. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used. In some examples, the one or more homologous nucleic acids may differ from the target nucleic acid by only up to 5 nucleotides (e.g., 4, 3, 2, or 1).

The assay methods may be applied in a diagnostic/prognostic setting to detect the presence or measure the level of a nucleic acid biomarker (e.g., a microRNA) that is associated with a target disease. For example, the methods may be used to detect/measure a specific microRNA, which may be associated with a specific disease, e.g., cancer. The methods can be used in detecting such a nucleic acid biomarker in subjects that are absent of any symptom of the disease for early stage diagnosis. The assay methods can also be used to detect nucleic acids of microorganisms for determining whether a subject has been infected by such microorganisms, for example, viruses (e.g., HBV, HCV, HPV, and HIV). The assay methods can also be applied to monitor an individual drug response. For example, the methods may be used to detect/measure a specific RNA, which may be associated with a specific drug toxicity or drug resistance event.

Those skilled in the art would have known that the application of the ultrasensitive assay methods described herein are not limited to diagnosis/prognosis purposes; such methods can be used to detect nucleic acids of interest for any purposes, for example, for research purposes. In some examples, the assay methods can be applied to detect a nucleic acid such as a microRNA in studies of its biological functions or in studies of biopathways in which the nucleic acid is involved. Alternatively, the assay methods described herein can also be used in development of nucleic acid-based therapeutic agents.

### III. Kits for Performing the Ultrasensitive Assay Methods

The present disclosure also provides kits for use in performing any of the assay methods described herein. Such kits may be designed for diagnostic uses or for other purposes, for example, research uses.

The kit described herein may include one or more containers housing components for performing the assay methods described herein and optionally instructions of uses. Specifically, such a kit may include one or more agents described herein (for example, a PNA probe, which maybe immobilized on a support member such as a bead, a suitable modified base, and a binding moiety that is conjugated to a detectable label plus ancillary chemicals and buffers), along with instructions describing the intended application and the proper use of these agents. In certain embodiments, the kit may be suitable for a diagnostic purpose. For example, the kit may contain apparatus for sample collection from a patient, and/or reagents for detecting diseases associated nucleic acid molecules. Kits for research purposes may contain the components in appropriate concentrations or quantities for running various experiments.

The kit described herein may contain an abasic PNA probe, which may be immobilized in a support member as described herein. Alternatively, the kit may contain the PNA probe in free form, the support member, and reagents necessary for linking the PNA probe onto the surface of the support member. For example, the support member in the kit may comprise chemical reactive moieties for the covalently linking of the PNA probes. Further, the kit comprises a modified base as described herein. In some embodiments, the modified base is conjugated to a first binding moiety as described herein. The kit may further comprise a second and/or a third binding moiety which is conjugated to a detectable label.

Any of the kits described herein may further comprise components needed for performing the assay methods. For example, it may contain components for use in detecting a signal released from the detectable label, directly or indirectly. In some examples, the detection step of the assay methods involves enzyme reaction, the kit may further contain the enzyme (e.g., β-galactosidase) and a suitable substrate.

Each of the components of the kits, where applicable, may be provided in liquid form (e.g., in solution), or in solid form, (e.g., a dry powder). In certain cases, some of the components may be constitutable or otherwise processable (e.g., to an active form), for example, by the addition of a suitable solvent or other species (for example, water or certain organic solvents), which may or may not be provided with the kit.

In some embodiments, the kits may optionally include instructions and/or promotion for use of the components provided. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which can also reflects approval by the agency of manufacture, use or sale for animal administration. As used herein, “promoted” includes all methods of doing business including methods of education, hospital and other clinical instruction, scientific inquiry, drug discovery or development, academic research, pharmaceutical industry activity including pharmaceutical sales, and any advertising or other promotional activity including written, oral and electronic communication of any form, associated with the invention. Additionally, the kits may include other components depending on the specific application, as described herein.

The kits may contain any one or more of the components described herein in one or more containers. The components may be prepared sterilely, packaged in syringe and shipped refrigerated. Alternatively it may be housed in a vial or other container for storage. A second container may have other components prepared sterilely. Alternatively the kits may include the active agents premixed and shipped in a vial, tube, or other container.

The kits may have a variety of forms, such as a blister pouch, vials, a shrink wrapped pouch, a vacuum sealable pouch, a sealable thermoformed tray, or a similar pouch or tray form, with the accessories loosely packed within the pouch, one or more tubes, containers, a box or a bag. The kits may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kits may also include other components, depending on the specific application, for example, containers, cell media, salts, buffers, reagents, syringes, needles, pipettes, a fabric, such as gauze, for applying or removing a disinfecting agent, disposable gloves, a support for the agents prior to administration etc.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1: Detection and Quantification of microRNA Using Single Nucleobase Labelling in Combination with Simoa Assay

An abasic peptide nucleic acid probe (PNA), containing a reactive amine instead of a nucleotide at a specific position in the sequence, for microRNA was conjugated to superparamagnetic beads. These beads were incubated with a sample containing microRNA, a biotinylated reactive nucleobase that was complementary to the missing base in the probe sequence, and a reducing agent. When a target molecule with an exact match in sequence hybridized to the capture probe, the reactive nucleobase was covalently attached to the backbone of the probe by a dynamic covalent chemical reaction. Single molecules of the biotin-labeled probe were then labeled with streptavidin-β-galactosidase (SβG), and the beads were loaded into an array of femtoliter wells, and sealed with oil. The array was imaged fluorescently to determine which beads were associated with single enzymes, and the average number of enzymes per bead was determined. The assay had a limit of detection of 500 fM, approximately 500 times more sensitive than a corresponding analog bead-based assay. This assay was used to measure microRNA-122 (miR-122), a biomarker of liver toxicity, extracted from the serum of patients who had acute liver injury due to acetaminophen, and control healthy patients. All patients with liver injury had higher levels of miR-122 in their serum compared to controls, and the concentrations measured correlated well with those determined using PCR. This approach allows rapid quantification of circulating microRNA with high sensitivity and specificity, and a limit of quantification suitable for clinical use. With further development, this method could facilitate translation of circulating miRNA from research tools into clinical biomarkers.

### Materials and Methods

### Materials

RNA target molecules were purchased desalted from Integrated DNA Technologies. All chemicals were obtained from SigmaAldrich and used as received. 2.8-μm-diameter superparamagnetic beads presenting carboxylic acid groups (Dynabeads® M-270), and resorufin-β-D-galactopyranoside (RGP) were obtained from ThermoFisher Scientific. Streptavidin-3-galactosidase (SβG) was conjugated using methods previously described. Rissin et al., Nat. Biotechnol. 2010, 28, 595-599. Simoa disks comprised of 24 arrays of 216,000 50-μL-sized microwells molded into cyclic olefin copolymer (COC) and bonded to a microfluidic manifold were obtained from Stratec Consumables. Kan et al., Lab Chip, 2012, 12, 977-985. Fluorocarbon oil (Krytox®) was obtained from DuPont. Concentrations of stock solutions of RNA and DNA were checked using a ThermoFisher NanoDrop 1000 Spectrophotometer.

### Synthesis of Capture Probe and Aldehyde-Modified, Biotinylated Nucleobase

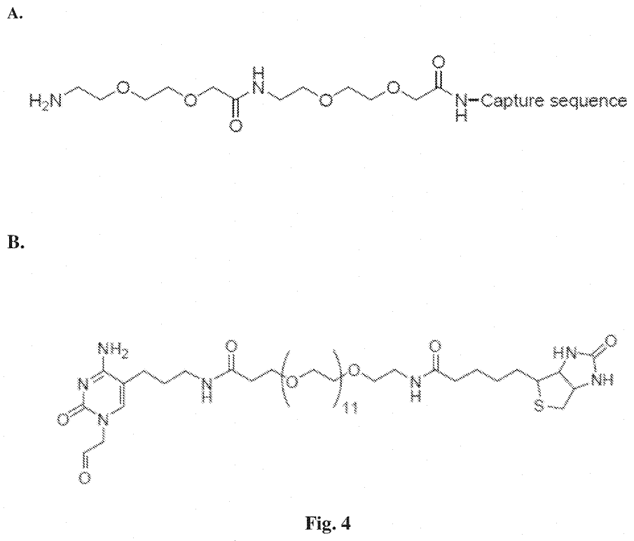

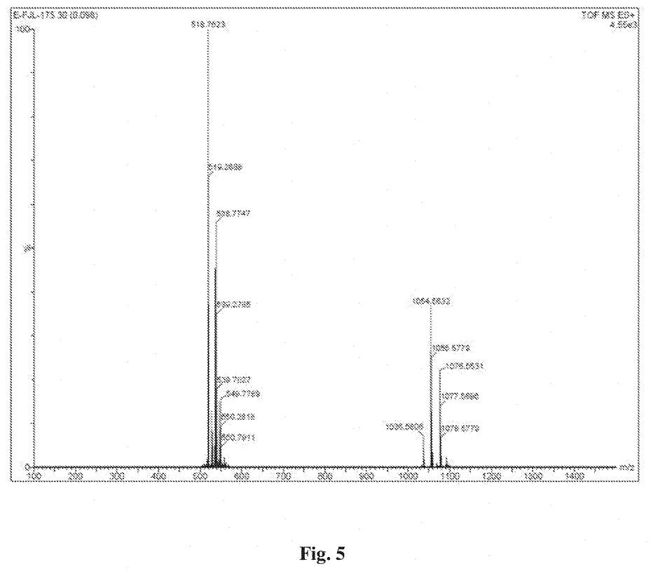

A peptide nucleic acid (PNA) probe containing an abasic “blank” position (where the corresponding base is missing) and terminated with an amino-PEG linker was synthesized using standard solid phase chemistry on a MultiPep Synthesiser (Intavis AG GmbH, Germany). The sequence of the probe (1) was designed to allow anti-parallel hybridization with the mature miR-122 target (2). The probe contained 3 thymidine bases modified with propionic acid side chains to increase its negative charge. The sequence of the probe and target are shown in Table 1 below. The structure of an exemplary amino-PEG linker is shown in FIG. 4, panel A. Aldehyde-modified cytosine, tagged with biotin via a 12 ethylene glycol units spacer is shown in FIG. 4, panel B, and was prepared using a synthetic route previously described. Venkateswaran et al., Talanta, 2016, 161, 489-496; and Bowler et al., Chem. Int. Ed. 2010, 49, 1809-1812.

TABLE 1
Sequences of capture probe and miR-122 target.

| SEQ | | |
|---|---|---|
| ID | | Peptide with abasic |
| NO: | Name | position (N′-C′) |
| 1 | Capture probe | xx-CACCAT*TGT*_ACACT*CCA |
| | | miRNA sequence (5′-3′) |
| 2 | Target miR-122 | UGGAGUGUGACAAUGGUGUUUG |

Key: xx = amino-PEG linker (FIG. 4, panel A); T* = thymidine containing a propanoic acid side chain at the gamma position; “_” = abasic “blank” monomer presenting a secondary amine; the italicized bases in 2 form a duplex with the capture probe, and G at position 9 (bold) is opposite the “blank” monomer and binds to the aldehyde-modified cytosine.
The mature sequence of miRNA-122 is 22 bases long. The
probe targets 18 bases out of the 22, leaving out the 4 bases at the 3′-end.

### Preparation of Superparamagnetic Beads Presenting Probes

100 μL of superparamagnetic beads (containing 2×10⁸ beads) were washed by adding 100 μL 0.01 M NaOH and mixing. The beads were pelleted, supernatant removed, and the beads were washed once in 100 μL 0.01 M NaOH and three times in 100 μL distilled water. The beads were then resuspended in 150 μL of freshly-prepared 50 mg/mL EDC in water, and incubated with slow tilt rotation at 23° C. for 30 min. After activation with EDC, the beads were washed once with 100 μL cold water and once with 100 μL cold MES buffer (50 mM, pH 5.0). 100 μL of a solution containing 1400 pmol capture probe (Table 1) and 700 pM of an amino-terminated diluent DNA sequence (5′-H₂N-GAGTGTTAGCTGTGAG-3;′ (SEQ ID NO: 6)) in MES buffer (50 mM, pH 5) was added to the activated beads. The mixture of probe and beads was incubated at 4° C. for 3 h with slow tilt rotation, and then washed with 50 mM MES buffer (pH 5). The remaining activated carboxyl groups were quenched by incubating the beads with 100 μL of 50 mM ethanolamine in PBS (pH 8) for 1 h, followed by three washes with 100 μL of 10% PEG10K and 0.1% Tween-20 in PBS. The capture beads were stored at 4° C. in 100 μL of 10% PEG10K and 0.1% Tween-20 in PBS.

Determination of miR-122 Concentration Using Quantitative Real-Time PCR (qRT-PCR)

PCR was performed using a leading commercial kit (Qiagen) and a procedure that has been described in detail elsewhere. Kroh et al., Methods 2010, 50, 298-301. cDNA was generated by reverse transcription using the miScript RT II kit (Qiagen) on 1.5 μL of RNA purified from serum (reaction volume=20 μL). This step includes the use of a poly(A) polymerase to poly-adenylated the target miRNAs, so that the molecule was sufficiently long to be converted into cDNA. 200 μL of RNase-free water was added to the sample containing cDNA, and 1 μL of this solution ([cel-miR-39-3p]=2.7×10⁵ copies/μL) was added to the PCR reaction mixture (reaction volume=25 μL). Quantitative PCR analysis was performed using the miScript SYBR Green PCR Kit (Qiagen) on a 7900HT Fast Real-Time PCR System (Applied Biosystems, CA), with an initial activation step of 95° C. for 15 min followed by 40 cycles of 3-step cycling (Denaturation: 15 s at 94° C.; Annealing: 30 s at 55° C.; Extension: 30 s at 70° C.). Ct values were determined for miR-122 and miR-39 in separate PCR reactions.

A calibration curve of Ct as a function of miRNA was determined by performing PCR on miR-39 at 5×10⁵, 5×10⁴, 5×10³, and 5×10² copies/L (independent from serum samples) according to manufacturer's protocol (miRNeasy Serum/Plasma Spiked-In Control, Qiagen). This calibration curve was used to determine the recovery of miR-39 from the samples based on the 10,800 copies/μL spiked control in the final PCR reaction. The calibration curve was also used to determine the concentration of miR-122 in each sample following the protocol reported in the miRNeasy Serum/Plasma Handbook. Concentrations of miR-122 were also corrected for the recovery measured for miR-39.

Determination of miR-122 Concentration Using Simoa Assay

Sample hybridization and labeling of the capture probe on the beads was conducted in a 96-well conical bottom microtiter plate (ThermoFisher Scientific; Cat No. 249944). The total volume of the labeling reaction was a 50 μL, containing 7.5 μL of sample or calibrator, 4,000,000 capture probe beads, 5 μM aldehyde-modified biotinylated cytosine (FIG. 4, panel B), 1 mM sodium cyanoborohydride, 0.1% SDS, and 10% w/v PEG-10K, in a buffered solution of 30 mM trisodium citrate and 300 mM sodium chloride, pH adjusted to 6.0 using HCl. The microtiter plate was placed on an incubating shaker (VWR; Cat No. 12620-930), set to 40° C., and mixed at 1000 rpm for 1 h. The beads were then pelleted on a custom-made 96-well plate magnet (VP Scientific), and washed 3 times with 230 μL of PBS and 0.1% Tween 20, followed by resuspension of the beads in 230 μL of PBS and 0.1% Tween 20.

Beads were analyzed on a Simoa HD-1 Analyzer (Quanterix Corporation). Wilson et al., J. Lab. Autom. 2016, 21, 533-47. The microtiter plate containing the labeled beads were loaded onto the instrument, along with enzyme conjugate and enzyme substrate (SβG and RGP, respectively), and consumables (Simoa disks, pipette tips, and reaction cuvettes). The Simoa measurements occurred in two steps on this instrument: liquid handling for enzyme labeling of the biotin-labeled probe beads; and, imaging and image analysis of the beads sealed in the Simoa disk. 100 μL of the solution containing beads in the microtiter plate was pipetted from the well into a reaction cuvette by a disposable tip pipettor. 100 μL of a solution containing 500 pM of SβG was added by a fixed tip pipettor to the reaction cuvette, the cuvette was shaken to disperse the beads, and incubated for 5 min. The beads were then magnetically separated and washed six times in 5×PBS and 0.1% Tween 20, and washed once in PBS. After aspiration of PBS, 25 μL of 100 μM RGP in PBS was added by a disposable tip pipettor to the reaction cuvette, the beads were mixed, and 15 μL of this bead-substrate mixture was transferred into an inlet port of an array on a Simoa disk. The beads were pulled by vacuum over the array of femtoliter wells, and the beads settled into the array of femtoliter wells. The beads were sealed with oil, and the single enzyme signal was generated over 30 s. The arrays were fluorescently imaged at multiple wavelengths at submicron resolution as described previously. Wilson et al., Lab. Autom. 2016, 21, 533-47. These fluorescent images were used to identify the location of beads within the wells, and the enzyme activity associated with each bead. Image analysis software on the instrument determined the average number of enzymes per bead (AEB). Rissin, et al. Anal. Chem. 2011, 83, 2279-2285. From the resulting AEB values of calibrators of known concentration, the concentrations of miR-122 in samples of unknown concentration were determined from interpolation using linear curve fitting in Microsoft Excel. Each sample was analyzed in duplicate to provide a mean AEB and a standard deviation.

### Results

### Simoa Assay Design

A schematic diagram of an assaying combining the single base labeling and Simoa developed for detecting miR-122 is shown in FIG. 1. The Simoa assay involves a specific 18-mer abasic peptide nucleic acid (PNA) probe that was complementary to miR-122. The cytosine base at the 9th position from the C-terminus of the PNA probe was replaced with a secondary amine group to yield a “blank” position in the capture sequence. Thymidine bases (3 total) in the PNA probe were modified with propionic acid at their gamma positions to improve hybridization efficiency (Rissin, et al. Anal. Chem. 2011, 83, 2279-2285), and the N-terminus of the probe was a primary amine.

The probe was covalently attached via its N-terminal amine to superparamagnetic beads presenting surface carboxyl groups. These beads were then incubated with a solution containing the sample, an aldehyde-modified cytosine base that contains biotin (FIG. 4, panel B), and a reducing agent, in reaction buffer with a total volume of 50 μL. If miR-122 was present in the sample then it hybridized to the complementary capture probe on the beads (FIG. 1, panel I). As the target sequence (SEQ ID NO: 2) has a guanine at the 9th position from the 5′-end that lines up with the “blank” position in the capture probe, the cytosine base binds to this guanine and places the aldehyde in close proximity to the secondary amine on the probe backbone. The aldehyde and amine reacted to form a stable iminium complex that was then reduced to a tertiary amine by reductive amination, thereby covalently incorporating biotin into the probe attached to the beads. Molecules with the same sequence but a single base mis-match at the 9th position, hybridize but a stable iminium group did not form and biotin was not incorporated (FIG. 1, panel II). Non-complementary sequences do not hybridize to the capture probe and biotin was not incorporated (FIG. 1, panel III).

The incorporated biotin labels were then labeled with an enzyme, streptavidin-(3-galactosidase (SβG). At low concentrations of miR-122, the ratio of enzyme labels to beads was <1, so that the distribution of enzymes on the beads followed a Poisson distribution [8], and single miR-122 molecules were labeled. These enzyme-labeled miR-122 molecules were detected by loading the beads into arrays of 216,000 microwells in the presence of a fluorogenic substrate of β-galactosidase. The wells were sealed with oil, so that the product of the enzyme-substrate reaction was confined to a small volume (˜50 fL). Single beads and associated enzyme activity in the wells were measured using a fluorescent imager with sub-micron resolution. The fraction of active beads and average number of enzymes per bead (AEB) were then determined as previously described. Bland et al., Lancet 1986, 1, 307-10.

### Sensitivity and Specificity of the Single Base Labeling-Simoa Assay

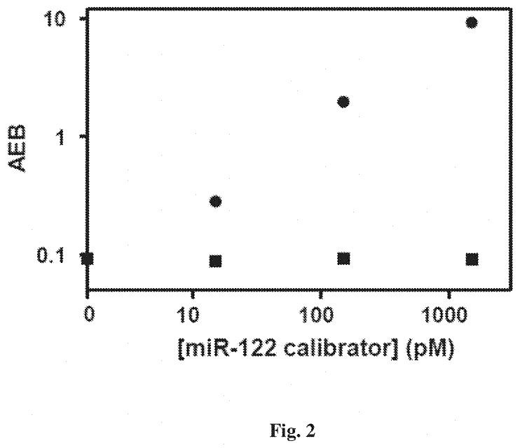

The analytical sensitivity of the Simoa assay was measured as the limit of detection (LOD) of miR-122. AEB values for buffered solutions spiked with known concentrations of a synthetic calibrator for miR-122 ranging from 0 to 1500 pM were determined using the Simoa assay (FIG. 2 and Table 2). The LOD of miR-122 using the Simoa assay was 500 fM.

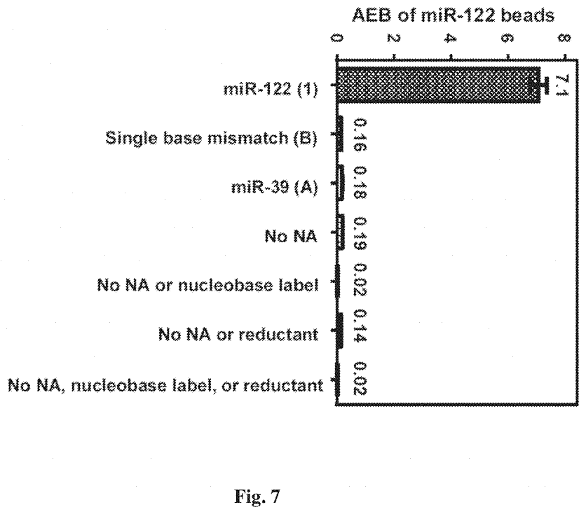

The specificity of the Simoa assay was determined using two nucleic acid molecules (Table 2). First, a random, off-target miRNA molecule (miR-39) was tested. AEB values for the off-target miRNA were at background (FIG. 7). Second, a molecule that differed in sequence from miR-122 by a single base at the complement to the labeling position was tested. AEB values for the single base mismatch miR-122 did not increase significantly over background (FIG. 2). Concentrations up to 15 μM of the single based mismatched molecule were also tested with no increase in signal above background (Table 3). Based on these data, the analytical specificity of the assay to a single-base mismatch was >3×10⁷-fold.

TABLE 2
Nucleic acid sequences of synthetic
calibrators for miR-122.

| SEQ | | |
|---|---|---|
| ID | | |
| NO: | Name | Sequence (5′-3′) |
| 3 | Calibrator for miR-122* | TGGAGTGTGACAATGGTGTTTG |
| 4 | Calibrator for single | TGGAGTGTTACAATGGTGTTTG |
| | base mismatch miR-122* | |
| 5 | Off-target miR-39^{†} | UCACCGGGUGUAAAUCAGCUUG |

*DNA analogs were used as calibrators for measuring miRNA in the clinical samples. DNA enabled long term storage of calibrators and more reliable determination of miR-122 concentration in samples. It was previously shown that uracil and thymidine have similar efficiencies for specifically templating the dynamic covalent chemical reaction between aldehyde-modified cytosine and secondary amine of the abasic PNA probe upon duplex formation (Bowler Ph.D. Dissertation, University of Edinburgh, UK, 2011). Equivalent performance of RNA and DNA calibrators for miR-122 in the assay presented here was determined (data not shown).
^{†}miRNeasy Serum/Plasma Spike-In Control (Cat No./ID: 219610, Qiagen): 10 pmol lyophilized C. elegans miR-39 miRNA mimic [5].

TABLE 3
AEB values for single base mismatch miR-122 from 0 pm to 15 mM.

| Concentration of | | | | | Signal-to-background |
|---|---|---|---|---|---|
| mismatch miR-122 | | Mean AEB | SD | CV | ratio |
| 15 | μM | 0.086 | 0.002 | 2% | 0.92 |
| 1.5 | μM | 0.071 | 0.004 | 6% | 0.76 |
| 150 | nM | 0.079 | 0.016 | 20% | 0.84 |
| 15 | nM | 0.083 | 0.014 | 17% | 0.89 |
| 1.5 | nM | 0.091 | 0.009 | 10% | 0.98 |
| 150 | pM | 0.093 | 0.007 | 7% | 1.00 |
| 15 | pM | 0.089 | 0.004 | 4% | 0.96 |
| 0 | pM | 0.093 | 0.009 | 9% | 1.00 |

(background)

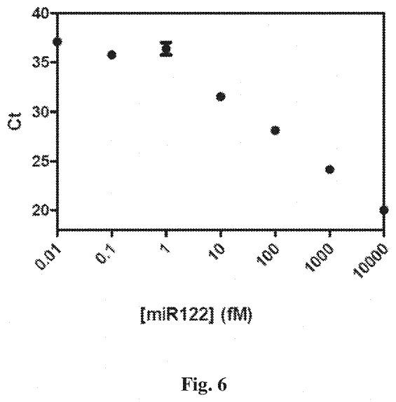

These results demonstrate that the single-base labeling/Simoa assay provided herein is both sensitive and specific for miR-122. The LOD of miR-122 obtained using the Simoa assay was compared to the LOD of miR-122 determined using a PCR kit that is widely used to measure microRNA. Kroh et al., Methods, 2010, 50, 298-301. The LOD of the PCR assay was determined by measuring the Ct value of buffer solutions spiked with known concentrations of the synthetic calibrator for miR-122 ranging from 0 to 10,000 fM (Table 4). The LOD of miR-122 using the PCR assay was 2.6 fM as calculated from a plot of Ct values as a function of miR-122 concentration (FIG. 6). Previous reports provided that the LOD of miR-122 using a conventional, analog bead-based assay was 300 pM. Rissin, et al. Anal. Chem. 2011, 83, 2279-2285.

TABLE 4
Ct values determined using PCR as a function
of the concentration of miR-122.

| Concen- | | | | | | |
|---|---|---|---|---|---|---|
| tration | | | | Mean | | |
| (fM) | Ct values | | | Ct | SD | CV |
| 0 | 37.10841 | n.d. | n.d. | 37.108 | n.d | n.d |
| 0.1 | 35.75835 | n.d. | n.d. | 35.75835 | n.d. | n.d. |
| 1 | 35.65074 | 36.61235 | 36.83607 | 36.366 | 0.630 | 1.7% |
| 10 | 31.58425 | 31.55038 | 31.44276 | 31.526 | 0.074 | 0.2% |
| 100 | 28.31135 | 27.97488 | 28.05834 | 28.115 | 0.175 | 0.6% |
| 1,000 | 24.08295 | 24.15437 | 24.16391 | 24.134 | 0.044 | 0.2% |
| 10,000 | 19.94744 | 19.8787 | 20.23545 | 20.021 | 0.189 | 0.9% |

The concentration is the concentration of miR-122 in the reverse transcription (RT) reaction. One RT reaction was carried out, and PCR performed on three aliquots from the RT reaction.
n.d. = not determined.

Taken together, results from this study show that the single base labeling-Simoa assay provides fM range detection of miR-122, which is similar to miR-122 concentrations that can be detected using RT-PCR. The fM range limit of detection is approximately 500 times more sensitive than a corresponding analog bead-based assay.

### Example 2: Clinical Validation of Single Base Labelling-Simoa Assay for Ultrasensitive Detection and Quantification of MiR-122 in Serum

The Simoa assay provided herein allows detection of short nucleic acids with high specificity using just a single probe, rather than multiple probes and primers used in most approaches for measuring miRNA. This specificity resulted from specific hybridization between the target and probe sequences, and incorporation of a single label. The use of a single probe greatly simplifies the measurement of short sequences of nucleic acids by simplifying probe design, and reducing the number of interactions that need to be screened for cross-reactivity in multiplex assays.

The Simoa assay provided herein is a sensitive and specific assay for detection of nucleic acids (e.g., miR-122). The Simoa assay may be used for detection of any nucleic acid biomarkers, such as nucleic acid biomarkers of liver toxicity, cancer, and sepsis. Alternatively, or in addition to, the Simoa assay provided herein may be used for detection of interfering nucleic acid therapies, and measurement of guide RNA used for gene editing systems, such as CRISPR/Cas9.

### Materials and Methods

Patients with Liver Injury

Samples from 4 patients (Patient Identifiers 3-4, 6, and 9) with acetaminophen (APAP) induced liver injury were recruited as part of the Markers and Paracetamol Poisoning Study (MAPP). Adult patients (16 years old and over) were recruited to the MAPP study if they fulfilled the study inclusion and exclusion criteria. Full informed consent was obtained from every participant and ethical approval for this study was from the South-East Scotland Research Ethics Committee. The inclusion criteria were: a history of APAP overdose that the treating clinician judged to warrant treatment with intravenous acetylcysteine as per the contemporaneous UK guidelines; the first blood sample collected within 24 hours of last APAP ingestion; and, the patient has capacity to consent. Patients were excluded if any of the following applied: patient detained under the Mental Health Act (UK); patient has known cognitive impairment; inability to provide informed consent for any reason; or an unreliable history of overdose. Patients having taken a single acute APAP overdose were recruited at the Royal Infirmary of Edinburgh, UK. The demographic details and clinical chemistry measurements performed on the patients are shown in Table 5.

TABLE 5
Demographics and clinical chemistry results for patients with liver injury.

| | | | | | Serum | | |
|---|---|---|---|---|---|---|---|
| | Age | | ALT activity | | Creatinine | ALP activity | Bilirubin |
| Patient | (yrs) | Sex | (U/L) (ULN 50) | INR | (μmol/L) | (U/L) | (μmol/dL) |
| 3 | 56 | M | 2150 | 1.3 | 56 | 141 | 23 |
| 4 | 23 | F | 487 | 1.5 | 60 | 64 | 14 |
| 6 | 46 | M | 2963 | 1.4 | 62 | 139 | 23 |
| 9 | 63 | F | 726 | 1.2 | 68 | 169 | 14 |

### Healthy Volunteers

The study was approved by the local research ethics committee (East Midlands—Nottingham 1 Research Ethics Committee), and performed in accordance with the Declaration of Helsinki. Informed consent was obtained from all participants. A total of 4 adults (23-42 years old) were recruited to this study (Patient identifiers HV1-4). Healthy volunteers were eligible if they had no history of liver disease, they were taking no medications, and they were willing to give blood samples by venepuncture.

### Collection and Preparation of Clinical Samples

For both healthy volunteers and patients with liver injury, blood samples were immediately centrifuged at 11,000×g for 15 min at 4° C., after which serum was separated into aliquots and frozen at −80° C.

### Control Sample Preparation

Serum from a healthy donor, who was negative for acute liver failure, was used for spike-in experiments. The blood from the donor was centrifuged at 11,000×g for 15 min at 4° C. after which serum was separated into 6 aliquots. A synthetic calibrator for miR-122 (SEQ ID NO: 3; Table 2) at different concentrations were added to each aliquot to yield 6 control samples (Control samples 1 to 6 containing: 10 nM, 10 nM, 1 nM, 1 nM, 100 pM, and 10 pM, respectively). These samples were frozen at −80° C. and subsequently processed in an identical fashion as the clinical samples.

RNA Isolation from Serum Samples

RNA was isolated from serum using the miRNeasy Serum/Plasma kit (Qiagen) according to manufacturer's protocol. Briefly, 500 μL QIAzol lysis reagent was added to 100 μL thawed serum, mixed, and incubated at 23° C. for 5 min. To calibrate the concentration of miR-122 and as a control to determine the efficiency of recovery and reverse transcription of miRNA, 3.5 μL of cel-miR-39-3p (1.6×10⁸ copies/pL) was added to each sample, in addition to 100 μL chloroform. Following shaking, incubation, and centrifugation, the upper aqueous phase was transferred, and 450 μL of ethanol was added and transferred to the RNeasy MinElute column. The column was washed with buffers RWT and RPE from the miRNeasy kit, and 80% ethanol, followed by drying and elution in 14 μL RNasefree water; total miRNA was recovered in ˜12 μL of water ([cel-miR-39-3p]=4×10⁷ copies/L). Quantification and quality assessment of small RNA, including the miRNA fraction, were performed using the Small RNA Assay kit (Agilent Technologies). Purified RNA was stored at <−80° C. before analysis with Simoa and RT-PCR.

### Results

### Clinical Validation

Clinical validation of the single base labeling-Simoa assay for detecting miR-122 in patient serum was performed using the serum of patients who had overdosed on acetaminophen and sustained clinically significant liver injury, and in the serum of healthy individuals.

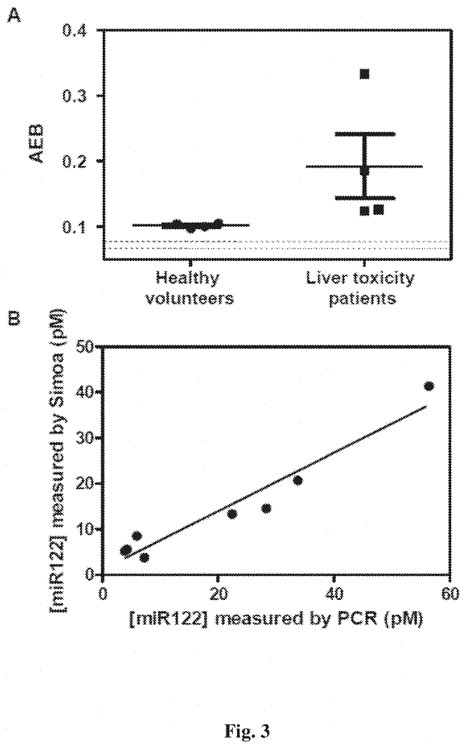

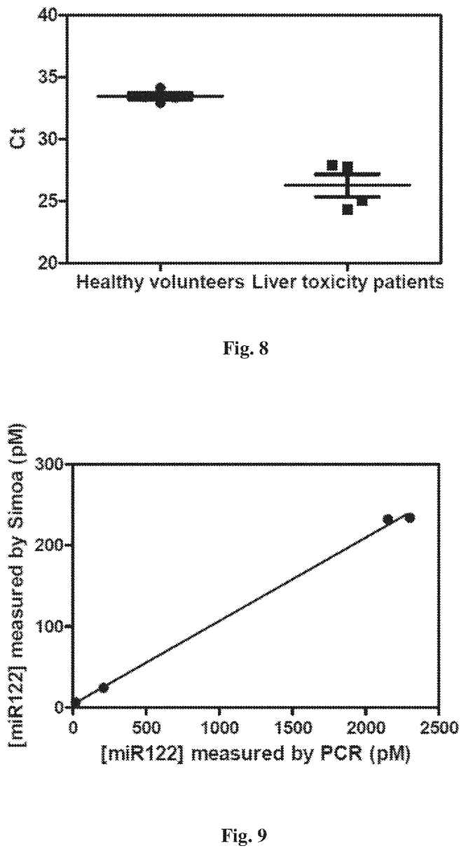

Concentrations of miR-122 in clinical samples and serum control samples spiked with synthetic miR-122 were determined using the Simoa assay (Table 6) and RT-PCR (Table 7). AEB values for samples from healthy volunteers were close together and slightly offset from the background AEB values, and AEB values for samples from patients having liver toxicity were significantly above background AEB values (FIG. 3, panels A and B). Ct values for samples from healthy volunteers were near background, and Ct values for samples from patients having liver toxicity were significantly above background (FIG. 8).

TABLE 6
Concentration of miR-122 in clinical samples determined using the Simoa assay.

| | | Sample | Concentration in | | | |
|---|---|---|---|---|---|---|
| | Sample | volume | 50 μL reaction | Recovery | Concentration in | Spiked |
| | number* | (μL) | (pM) | (%)^{†} | sample (pM) | concentration |
| Patient | | | | | | |
| ID | | | | | | |
| HV1 | 1 | 90 | 1.51 | 51 | 2.65^{‡} | |
| | 2 | 90 | 3.91 | 81 | 4.27^{‡} | |
| HV2 | 1 | 95 | 1.51 | 84 | 1.51^{‡} | |
| | 2 | 95 | 3.81 | 94 | 3.41^{‡} | |
| HV3 | 1 | 95 | 1.34 | 83 | 1.37^{‡} | |
| | 2 | 95 | 2.95 | 84 | 2.97^{‡} | |
| HV4 | 1 | 100 | 1.13 | 78 | 1.17^{‡} | |
| | 2 | 100 | 3.32 | 70 | 3.81^{‡} | |
| 3 | 1 | 105 | 2.85 | 41 | 5.28 | |
| | 2 | 105 | 6.46 | 58 | 8.54 | |
| 4 | 1 | 95 | 11.57 | 73 | 13.34 | |
| | 2 | 95 | 13.66 | 79 | 14.56 | |
| 6 | 1 | 95 | 19.67 | 80 | 20.71 | |
| | 2 | 95 | 31.43 | 64 | 41.35 | |
| 9 | 1 | 90 | 3.21 | 75 | 3.81 | |
| | 2 | 90 | 6.25 | 98 | 5.67 | |
| Controls | | | | | | |
| 1 | 1 | 100 | n.d. | 49 | n.d. | 10000 |
| 2 | 2 | 100 | n.d. | 67 | n.d. | 10000 |
| 3 | 3 | 100 | 162.66 | 56 | 232.13 | 1000 |
| 4 | 4 | 100 | 126.47 | 43 | 233.96 | 1000 |
| 7 | 5 | 100 | 13.78 | 45 | 24.31 | 100 |
| 8 | 6 | 100 | 3.69 | 44 | 6.70 | 10 |

n.d. = not determined
*For each patient and healthy volunteer, miRNA was extracted from two samples and its concentration was determined.
^{†}Recovery of miRNA from the sample was determined by PCR using miR-39 control spikes. The concentration in samples were corrected for this recovery.
^{‡}The AEB values determined for these samples were close together and slightly offset from the background AEB (FIG. 3, panels A and B). This observation could be explained by a systematic difference in the background signal of the sample matrix compared to the calibration buffer, rather than by specific measurement of miR-122. Without further experimental confirmation, we regard these samples as not quantified for miR-122.

TABLE 7
Concentration of miR-122 in clinical samples determined using the PCR assay.

| | | Volume | | Copies/μL | | | Concentration | |
|---|---|---|---|---|---|---|---|---|
| Patient | Sample | sample | | in PCR | | Recovery | in sample | Spiked |
| ID | number* | (μL) | Ct | reaction | Copies in sample | (%) | (pM) | concentration |
| HV1 | 1 | 90 | 34:4 | 76 | 3,345,710 | 51 | 0.12^{‡} | |
| | 2 | 90 | 34.1 | 92 | 4,050,941 | 81 | 0.09^{‡} | |
| HV2 | 1 | 95 | 32.4 | 281 | 12,355,108 | 84 | 0.26^{‡} | |
| | 2 | 95 | 32.9 | 200 | 8,783,626 | 94 | 0.16^{‡} | |
| HV3 | 1 | 95 | 33.3 | 156 | 6,875,551 | 83 | 0.15^{‡} | |
| | 2 | 95 | 33.4 | 147 | 6,461,253 | 84 | 0.13^{‡} | |
| HV4 | 1 | 100 | 33.2 | 169 | 7,419,621 | 78 | 0.16^{‡} | |
| | 2 | 100 | 33.4 | 152 | 6,705,783 | 70 | 0.16^{‡} | |
| 3 | 1 | 105 | 29.1 | 2,295 | 100,969,205 | 41 | 3.88 | |
| | 2 | 105 | 27.9 | 4,942 | 217,428,017 | 58 | 5.96 | |
| 4 | 1 | 95 | 25.6 | 21,300 | 937,196,939 | 73 | 22.41 | |
| | 2 | 95 | 25.1 | 29,113 | 1,280,986,047 | 79 | 28.31 | |
| 6 | 1 | 95 | 24.8 | 35,139 | 1,546,107,754 | 80 | 33.74 | |
| | 2 | 95 | 24.3 | 47,006 | 2,068,243,613 | 64 | 56.41 | |
| 9 | 1 | 90 | 27.4 | 6,702 | 294,868,894 | 75 | 7.24 | |
| | 2 | 90 | 27.8 | 5,120 | 225,300,348 | 98 | 4.24 | |

Controls

| | | | | | | | | |
|---|---|---|---|---|---|---|---|---|
| 1 | 1 | 100 | 14.5 | 23,245,908 | 1,022,819,938,047 | 49 | 34380.66 | 10000 |
| 2 | 2 | 100 | 14.5 | 23,790,920 | 1,046,800,483,612 | 67 | 25999.14 | 10000 |
| 3 | 3 | 100 | 18.7 | 1,653,095 | 72,736,170,424 | 56 | 2151.74 | 1000 |
| 4 | 4 | 100 | 19.0 | 1,363,925 | 60,012,704,821 | 43 | 2301.44 | 1000 |
| 7 | 5 | 100 | 22.7 | 130,673 | 5,749,590,955 | 45 | 210.31 | 100 |
| 8 | 6 | 100 | 26.4 | 12,485 | 549,318,183 | 44 | 20.67 | 10 |

*For each patient and healthy volunteer, miRNA was extracted from two samples and its concentration was determined.
^{†}Recovery of miRNA from the sample was determined by PCR using miR-39 control spikes. The concentration in samples were corrected for this recovery.
^{‡}The Ct values of samples from healthy volunteers were above or slightly below those of background samples (Table 3), so the detection of miR-122 in these samples is ambiguous.

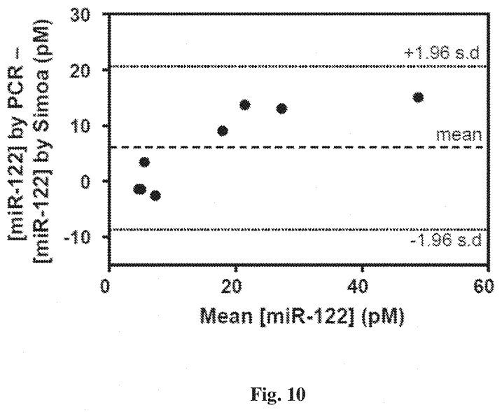

The concentrations of miR-122 in serum from patients determined using the Simoa assay were well correlated with those obtained using the PCR assay (FIG. 9). The Bland-Altman plot for concentrations of miR-122 in serum samples determined using the Simoa assay and the PCR assay showed that the miR-122 concentration as measured by the Simoa assay is lower than those obtained using the PCR assay with an average bias of 6.1 pM (95% limit of agreement=−8.5-20.8 pM) (FIG. 10).

In sum, these data indicate that the sensitivity and specificity of the Simoa assay was sufficient to measure miR-122 in patients, and showed a clear distinction between healthy controls and those with clinical liver toxicity after drug overdose. The sensitivity and specificity of the Simoa assay was also comparable to that of the RT-PCR assay.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A method for detecting a nucleic acid of interest in a sample, comprising:
(i) providing a sample suspected of containing a nucleic acid of interest;
(ii) providing a plurality of beads, on which a peptide nucleic acid (PNA) probe is immobilized, wherein the PNA probe comprises a nucleotide sequence that is complementary to the nucleic acid of interest or a portion thereof, and wherein the PNA probe lacks a base at a position corresponding to a target nucleotide in the nucleic acid of interest;
(iii) incubating the sample with the plurality of beads under conditions allowing for formation of a nucleic acid/PNA complex;
(iv) contacting the plurality of beads with a modified base that is complementary to the base of the target nucleotide in the nucleic acid of interest, wherein the modified base is conjugated to a first binding moiety and modified with a chemical group that reacts with the PNA probe;
(v) contacting the plurality of beads with a second binding moiety that binds the first binding moiety and comprises a detectable label; and
(vi) determining the fraction of beads associated with at least one detectable label, which is indicative of the level of the nucleic acid of interest in the sample.

2. The method of claim 1, wherein the first binding moiety comprises biotin and the second binding moiety comprises streptavidin.

3. A method for detecting a nucleic acid of interest in a sample, comprising:
(i) providing a sample suspected of containing a nucleic acid of interest;
(ii) providing a plurality of beads, on which a peptide nucleic acid (PNA) probe is immobilized, wherein the PNA probe comprises a nucleotide sequence that is complementary to the nucleic acid of interest or a portion thereof, and wherein the PNA probe lacks a base at a position corresponding to a target nucleotide in the nucleic acid of interest;
(iii) incubating the sample with the plurality of beads under conditions allowing for formation of a nucleic acid/PNA complex;
(iv) contacting the plurality of beads after with a modified base that is complementary to the base of the target nucleotide in the nucleic acid of interest, wherein the modified base is conjugated to a first binding moiety and modified with a chemical group that reacts with the PNA probe;
(v) contacting the plurality of beads with a second binding moiety that binds the first binding moiety;
(vi) contacting the plurality of beads with a third binding moiety that binds the second binding moiety and comprises a detectable label; and
(vii) determining the fraction of beads associated with at least one detectable label, which is indicative of the level of the nucleic acid of interest in the sample.

4. The method of claim 3, wherein the second binding moiety comprises an antibody specific to the first binding moiety.

5. The method of claim 4, wherein the antibody is biotinylated.

6. The method of claim 5, wherein the third binding moiety comprises streptavidin.

7. The method of any one of claims 3-6, wherein the first binding moiety is a fluorescein label.

8. The method of any one of claims 3-7, wherein the second binding moiety comprises a thiolated heterocarbon chain which react specifically with first binding moiety.

9. The method of claim 8, wherein the thiolated heterocarbon moiety is biotinylated.

10. The method of claim 9, wherein the third binding moiety comprises streptavidin.

11. The method of any one of claims 8-10, wherein the first binding moiety is a maleimide group.

12. The method of any one of claims 1-11, wherein the detectable label releases a signal directly.

13. The method of claim 12, wherein the detectable label is a dye or a fluorescent agent.

14. The method of any one of claims 1-11, wherein the detectable label releases a signal indirectly.

15. The method of claim 14, wherein the detectable label is a β-galactosidase.

16. The method of any one of claims 1-15, wherein the nucleic acid of interest is a microRNA.

17. The method of any one of claims 1-16, wherein the chemical group in the modified base is an aldehyde group, a ketone group, a thiol group, or a diol group.

18. The method of claim 1, wherein step (vi) is performed using single molecule arrays.

19. The method of claim 3, wherein step (vii) is performed using single molecule arrays.

20. The method of claim 8, wherein step (vii) is performed using single molecule arrays.

21. The method of any one of claims 1-20, wherein the sample is a biological sample obtained from a subject.

22. The method of claim 20, wherein the nucleic acid of interest is a microRNA associated with a target disease.

23. The method of claim 21, wherein the subject is suspected of having the target disease.

24. The method of any one of claims 1-22, wherein the PNA probe has 10-30 monomer units.


SEQUENCE LISTING (6 sequences)

SEQ ID NO: 1 - DNA, 18 bp, Artificial Sequence
  Synthetic Polynucleotide
  misc_feature (6)..(6): contains a propanoic acid side chain at the gamma position
  misc_feature (9)..(9): contains a propanoic acid side chain at the gamma position
  misc_feature (10)..(10): n is an abasic “blank” monomer presenting a secondary amine
  misc_feature (15)..(15): contains a propanoic acid side chain at the gamma position
caccattgtnacactcca

SEQ ID NO: 2 - RNA, 22 nt, Artificial Sequence
  Synthetic Polynucleotide
uggagugugacaaugguguuug

SEQ ID NO: 3 - DNA, 22 bp, Artificial Sequence
  Synthetic Polynucleotide
tggagtgtgacaatggtgtttg

SEQ ID NO: 4 - DNA, 22 bp, Artificial Sequence
  Synthetic Polynucleotide
tggagtgttacaatggtgtttg

SEQ ID NO: 5 - RNA, 22 nt, Artificial Sequence
  Synthetic Polynucleotide
ucaccggguguaaaucagcuug

SEQ ID NO: 6 - DNA, 16 bp, Artificial Sequence
  Synthetic Polynucleotide
gagtgttagctgtgag